(19) United States
(12) Patent Application Publication
(10) Pub. No.: US 2026/0294275 A1
(43) Pub. Date: Oct. 1, 2026

(54) METHODS FOR ASSESSING REGIONAL LUNG CHARACTERISTICS, AND RELATED SYSTEMS

(71) Applicant: Timpel Medical B.V., Eindhoven (NL)

(72) Inventor: Eduardo Leite Vieira Costa, São Paulo-SP (BR)

(21) Appl. No.: 19/635,001

(22) Filed: Mar. 31, 2026

Related U.S. Application Data
(60) Provisional application No. 63/780,882, filed on Mar. 31, 2025.
(60) Provisional application No. 63/932,245, filed on Dec. 5, 2025.

(51) Int. Cl. A61B 5/085; A61B 5/0536

(52) U.S. Cl. CPC A61B 5/085; A61B 5/0536

(57) ABSTRACT

A method for assessing regional lung compliance in a mechanically ventilated patient includes acquiring electrical impedance tomography data representative of a plurality of lung regions while a pressure-volume maneuver is performed on the mechanically ventilated patient, and associating pressure values from the pressure-volume maneuver with corresponding regional electrical impedance tomography data to obtain a regional pressure-impedance curve, determining the regional compliance at more than two different pressures, determining a pressure P_{maxC} at which the regional compliance is greatest, and determining an occurrence of at least one of overdistension at pressures above P_{maxC} and collapse at pressures below P_{maxC}. The method also includes calculating cumulative overdistension and cumulative collapse, and outputting a pressure P_{med} at which curves representing the cumulative overdistension and the cumulative collapse over the more than two different pressures intersect. Related systems and methods are also disclosed.

### CROSS-REFERENCE TO RELATED APPLICATIONS

This application claims the benefit under 35 U.S.C. § 119(e) of U.S. Provisional Patent Application Ser. No. 63/780,882, filed Mar. 31, 2025, and of U.S. Provisional Patent Application Ser. No. 63/932,245, filed Dec. 5, 2025, the disclosure of each of which is hereby incorporated herein in its entirety by this reference.

### TECHNICAL FIELD

Embodiments of the present disclosure generally relate to methods for assessing lung characteristics, in particular lung compliance. In particular, embodiments of the present disclosure relate to methods for assessing regional lung compliance and related systems, apparatus, and components.

### BACKGROUND

The treatment of patients with respiratory failure may utilize artificial ventilator support. Patients with respiratory failure, such as patients with acute respiratory distress syndrome (ARDS), may be exposed to artificial ventilator support having a defined positive end expiratory pressure (PEEP). Lung compliance is a measure of the lung's ability to stretch and expand in response to pressure changes. It is defined as the change in lung volume per unit change in transpulmonary pressure. In clinical settings, lung compliance is typically measured using pressure and flow sensors placed at the patient's airways. These sensors capture the pressure exerted by the ventilator and the flow of air into the lungs, allowing for the calculation of compliance by assessing the volume changes in the lungs as a function of pressure changes.

Maximizing lung compliance during mechanical ventilation ensures that the patient receives the required air volume with minimal pressure, reducing the risk of ventilator-induced lung injury. PEEP is an important component in this process, as it helps to recruit collapsed alveoli and keep them open, thereby preventing lung collapse. Both lung collapse and alveolar overdistension can cause or perpetuate lung injury. As more alveoli are open and functional, lower pressures are required to instill a certain volume of air.

However, titrating PEEP using only pressure and flow sensors does not account for the heterogeneity of the lung. Due to lung and disease heterogeneity, the airway pressure required to maintain some regions of the lung fully open frequently leads to significant alveolar overdistension in other regions.

During a decremental PEEP titration maneuver, there may be relief of overdistension in some regions of the lung while others may collapse progressively. The former may lead to increased lung compliance, while the latter may lead to decreased lung compliance. When monitoring the overall lung compliance during a PEEP titration maneuver, the net result of these two antagonistic phenomena usually produces a dampened, non-informative lung compliance curve, which can lead to suboptimal ventilation settings if not properly addressed.

### BRIEF SUMMARY

A method for assessing regional lung compliance in a mechanically ventilated patient is disclosed. The method includes acquiring electrical impedance tomography data representative of a plurality of lung regions while a pressure-volume maneuver is performed on the mechanically ventilated patient. The method further includes associating pressure values from the pressure-volume maneuver with corresponding regional electrical impedance tomography data to obtain a regional pressure-impedance curve, determining the regional compliance at more than two different pressures, determining a pressure P_{maxC} at which the regional compliance is greatest, and determining an occurrence of at least one of overdistension at pressures above P_{maxC} and collapse at pressures below P_{maxC}. The method also includes calculating cumulative overdistension and cumulative collapse based on the occurrence of overdistension and collapse at the plurality of lung regions at each of the more than two different pressures, and outputting a pressure P_{med} at which curves representing the cumulative overdistension and the cumulative collapse over the more than two different pressures intersect.

A system for assessing regional lung compliance in a mechanically ventilated patient is disclosed. The system includes an electrical impedance tomography acquisition system configured to acquire electrical impedance tomography data representative of a plurality of lung regions while a pressure-volume maneuver is performed on the mechanically ventilated patient, one or more sensors or interfaces configured to obtain pressure values from the pressure-volume maneuver, and a processor configured to associate the pressure values with corresponding regional electrical impedance tomography data to obtain a respective pressure-impedance curve, determine regional compliance at more than two different pressures, determine a pressure P_{maxC} at which the regional compliance is greatest, determine occurrence of at least one of overdistension at pressures above P_{maxC} and collapse at pressures below P_{maxC}, calculate cumulative overdistension and cumulative collapse based on the occurrence of overdistension and collapse at the plurality of lung regions at each of the more than two different pressures, and output a pressure P_{med} at which curves representing the cumulative overdistension and the cumulative collapse over the more than two different pressures intersect.

### BRIEF DESCRIPTION OF THE DRAWINGS

While the specification concludes with claims particularly pointing out and distinctly claiming embodiments of the present disclosure, the advantages of embodiments of the disclosure may be more readily ascertained from the following description of embodiments of the disclosure when read in conjunction with the accompanying drawings in which:

FIG. 1 is a schematic diagram of a portion of an Electrical Impedance Tomography (EIT) system showing a plurality of electrodes positioned around a patient's thorax, according to one or more embodiments of the present disclosure;

FIG. 2 is a schematic diagram showing a cross-section of the thorax of the patient along the plane of the electrodes according to one or more embodiments of the present disclosure;

FIG. 3 is a schematic block diagram of an EIT system, according to an embodiment of the disclosure;

FIG. 4 is a schematic view of a system for determining lung compliance, according to one or more embodiments of the disclosure;

FIG. 5 is a schematic diagram showing a cross-section of the thorax of the patient along the plane of the electrodes, according to embodiments of the disclosure;

FIG. 6 is a graph of impedance data and pressure measured at region of interest A of FIG. 5, according to embodiments of the disclosure;

FIG. 7 is a graph of impedance data and pressure measured at region of interest B of FIG. 5, according to embodiments of the disclosure;

FIG. 8 is a graph of impedance data and pressure measured at region of interest C of FIG. 5, according to embodiments of the disclosure;

FIG. 9 is a graph of cumulative overdistension and cumulative collapse, according to embodiments of the disclosure;

FIGS. 10A-10C are EIT images of lungs according to embodiments of the disclosure;

FIGS. 11A-11C are graphs depicting EIT and compliance data for ROI C;

FIGS. 12A-12C are graphs depicting EIT and compliance data for ROI A;

FIGS. 13A-13B are graphical representations of regional overdistension and collapse for ROI A and ROI C;

FIG. 14 is a graph illustrating cumulative collapse and cumulative overdistension for ROI A and ROI C; and

FIG. 15 is a graph illustrating an example of local compliance estimation using a sliding-window regression approach.

### DETAILED DESCRIPTION

Illustrations presented herein are not meant to be actual views of any particular material, component, or system, but are merely idealized representations that are employed to describe embodiments of the disclosure.

The following description provides specific details, such as material types, dimensions, and processing conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional fabrication techniques employed in the industry. In addition, the description provided below does not form a complete process flow, apparatus, or system for assessing regional lung characteristics, in particular lung compliance and identifying overdistension and collapse in a mechanically ventilated patient, or a related method. Only those process acts and structures necessary to understand the embodiments of the disclosure are described in detail below. Additional acts to assess regional lung compliance may be performed by conventional techniques. Further, any drawings accompanying the present application are for illustrative purposes only and, thus, are not drawn to scale. Additionally, elements common between figures may retain the same numerical designation. To easily identify the discussion of any particular element or act, the most significant digit or digits in a reference number refer to the figure number in which that element is first introduced or where the element is described in detail.

In the following detailed description, reference is made to the accompanying drawings, which form a part hereof, and in which is shown by way of illustration specific embodiments in which the disclosure may be practiced. These embodiments are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

In accordance with common practice, the various features illustrated in the drawings may not be drawn to scale. The illustrations presented herein are not meant to be actual views of any particular apparatus (e.g., device, system, etc.) or method, but are merely representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus or all operations of a particular method.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof. Some drawings may illustrate signals as a single signal for clarity of presentation and description. It should be understood by a person of ordinary skill in the art that the signal may represent a bus of signals, wherein the bus may have a variety of bit widths and the disclosure may be implemented on any number of data signals including a single data signal.

The various illustrative logical blocks, modules, and circuits described in connection with the embodiments disclosed herein may be implemented or performed with a general purpose processor, a special purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A general-purpose processor may be considered a special-purpose processor while the general-purpose processor executes instructions (e.g., software code) stored on a computer-readable medium. A processor may also be implemented as a combination of computing devices, e.g., a combination of a DSP and a microprocessor, a plurality of microprocessors, one or more microprocessors in conjunction with a DSP core, or any other such configuration.

Also, it is noted that embodiments may be described in terms of a process that may be depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a function, a procedure, a subroutine, a subprogram, etc. Furthermore, the methods disclosed herein may be implemented in hardware, software, or both. If implemented in software, the functions may be stored or transmitted as one or more instructions or code on computer-readable media. Computer-readable media include both computer storage media and communication media, including any medium that facilitates transfer of a computer program from one place to another.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “positive end-expiratory pressure” (PEEP) refers to the pressure maintained in the lungs at the end of expiration during mechanical ventilation.

As used herein, the term “driving pressure” refers to the difference between plateau pressure (e.g., the elevated pressure above the PEEP) used to inflate the lungs of the patient and positive end-expiratory pressure (PEEP).

As used herein, the term “collapse” and “collapsed” refer to the progressive collapse of lung regions (e.g., alveoli) that may occur during the deflation phase of a pressure-volume (PV) maneuver, wherein alveolar units close at varying pressures over time as a PV maneuver is performed, rather than at a single critical threshold. A collapsed lung region may require higher pressure differences to transition from the collapsed stage to a state where the region is configured to take on air and inflate in a compliant manner.

As used herein, the terms “overdistended,” and “overdistension” refer to the progressive overdistension (e.g., overstretching) of lung regions (e.g., alveoli) that may occur at higher pressure levels of a pressure-volume (PV) maneuver, where different lung units become overdistended at varying pressures rather than at a single critical threshold. Overdistended regions may be relieved during the deflation phase of a pressure-volume (PV) maneuver. Due to reduced compliance, an overdistended lung region may require higher pressure differences to take on a larger volume of air. Eventually, an overdistended lung region may become damaged.

As used herein, the term “regional overdistension” may refer to regions of the lung that exhibit a loss of compliance with higher pressures, or alternatively, regions that exhibit a gain in compliance with lower pressures.

As used herein, the term “regional collapse” may refer to regions of the lung that exhibit a loss of compliance with lower pressures, or alternatively, regions that exhibit a gain in compliance with higher pressures.

As used herein, the terms “compliance” and “compliant” refer to the relative ease with which a lung region (e.g., alveoli) expands in response to changes in pressure during a pressure-volume (PV) maneuver. Compliance may vary across different lung regions and at different points during inflation and deflation phases of a PV maneuver. A region with high compliance inflates readily with small changes in pressure, whereas a region with low compliance may resist inflation and exhibit reduced volume change for a given pressure difference.

As used herein, the term “electrical impedance data” or “impedance data” relates to data obtained from an electrical impedance tomography (EIT) system or an electrical impedance measurement device, or any other data that relates to electrical characteristics (e.g., impedance, resistivity, capacitance, inductance, permittivity, etc.) of the measured subject (e.g., a patient, an object). Electrical impedance data refers to raw (i.e., unprocessed) data, filtered data, and/or tomographically reconstructed data. Tomographic reconstructions from electrical impedance data may be specifically referred herein as “electrical impedance images” or “electrical impedance videos.” Electrical impedance images may be further described as “absolute impedance images,” “differential impedance images,” and “volumetric images” of a subject.

As used herein, the term “absolute impedance images” refer to images in which the representation (e.g., value) of each pixel or voxel is associated to the impedance of the corresponding portion of the subject being imaged.

As used herein, the term “differential impedance images” refers to images in which the representation (e.g., value) of each pixel or voxel is associated to a change of the impedance of the corresponding portion of the subject relative to a baseline image.

As used herein, the term “volumetric images” refers to images in which the representation (e.g., value) of each pixel or voxel is associated with a volume of a biological fluid (e.g., air, blood, water, tissue type). Moreover, it should be understood that the impedance images or videos, as referred herein, may be tomographic images that may be two-dimensional (2-D) or three-dimensional (3-D), as well as tomographic movies with a time dimension.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, the term “substantially” in reference to a given parameter, property, or condition means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, or at least about 99% met, or even at least about 100.0 percent met.

As used herein, “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

The Pressure-Volume (PV) maneuver is a technique used to assess lung characteristics. A PV curve is obtained by plotting the relationship between airway pressure and lung volume during a controlled inflation and deflation maneuver. The curve typically exhibits a sigmoidal shape, with lower and upper inflection points indicating the onset of alveolar recruitment and overdistension, respectively. The PV maneuver provides insights into global lung compliance but does not account for regional variations. Embodiments of the disclosure may facilitate greater accuracy in identifying regional lung compliance using EIT during the expiratory limb of the PV curve obtained during a PV maneuver. By analyzing the tangent to the expiratory limb of the PV curve, clinicians may assess regional compliance and identify areas of overdistension and collapse for the range of pressures covered during the Pressure-Volume (PV) maneuver, being a faster and simpler way to titrate PEEP than otherwise possible with conventional methods.

A method for assessing regional lung compliance and identifying overdistension and collapse in a mechanically ventilated patient may thus include applying a controlled pressure-volume (PV) maneuver to the patient between predetermined pressure limits, capturing electrical impedance tomography (EIT) data during the PV maneuver to generate impedance images corresponding to multiple lung regions. For each pixel or region of interest, a pressure-impedance (PZ) curve may be constructed, wherein impedance assessed at the lung region is used as a surrogate for regional volume change during the PV maneuver. From the expiratory phase of each PZ curve, a regional compliance curve for that region or pixel may be derived by determining a local slope of the pressure-impedance curve at at least two pressures within the range of the PV maneuver. In some embodiments, the local slope may be graphically represented by the angle α of a tangent to the curve, such that the tangent angle provides a graphical representation of the local slope and corresponding regional compliance value (Compliance_{z}). In some embodiments, impedance variation (ΔZ) in a region may be used as a surrogate for regional volume change during the PV maneuver. Regional compliance may be calculated according to the equation:

$${Compliance}_{ROI} = \frac{Δ Z}{{P}_{plateau} - PEEP}$$

where ΔZ refers to change in impedance, Pₚₗₐₜₑₐᵤ refers to plateau pressure, and PEEP refers to positive end-expiratory pressure.

In some embodiments, during a PV maneuver, regional compliance may be assessed as the slope of the pressure-impedance (PZ) curve in each region of interest. To determine this slope, a PZ curve may be constructed for each region or pixel using impedance data acquired during the PV maneuver. The impedance data may include raw impedance measurements at the region of interest, reconstructed tomographic images based on the impedance data, or filtered or modeled curves that fit the impedance data. The local slope of the PZ curve at a given pressure may be used as an estimate of regional compliance at that pressure. In some embodiments, the angle of a tangent to the curve may provide a graphical representation of the local slope and, therefore, of the regional compliance at that pressure.

For each pixel or region of interest, a pressure P_{maxC} may be identified at which maximum Compliance_{z} is attained. For pressures above P_{maxC}, a reduction in Compliance_{z} may indicate overdistension of the patient's alveoli within that region, and, for pressures below P_{maxC}, a reduction in Compliance_{z} may indicate collapse of the patient's alveoli in that region. Cumulative measures of overdistension and collapse may be calculated considering multiple regions based on the respective decreases in Compliance_{z} at pressures above and below P_{maxC}. Graphical representations of the cumulative overdistension and collapse for the lung regions may be produced to facilitate a user's selection of an appropriate PEEP level to ensure a compromise between alveoli collapse and overdistension, so the patient receives sufficient air with minimal risk of lung injury that can be caused both by collapse and overdistension. Graphical representations may be produced to indicate regional overdistension and collapse at each pressure of the PV maneuver at which regional compliance was assessed. In other words, the methods and systems according to embodiments of the disclosure may facilitate a reduction in driving pressure (i.e., the difference between plateau pressure and PEEP) during mechanical ventilation by facilitating selection of PEEP levels in which the best compromise between collapse and overdistension is obtained. By selecting a PEEP level that maximizes alveolar recruitment while minimizing overdistension, the disclosed approach stabilizes lung mechanics, maintains consistent and homogeneous tidal volume delivery, and reduces the overall driving pressure, thereby mitigating risks associated with mechanical ventilation.

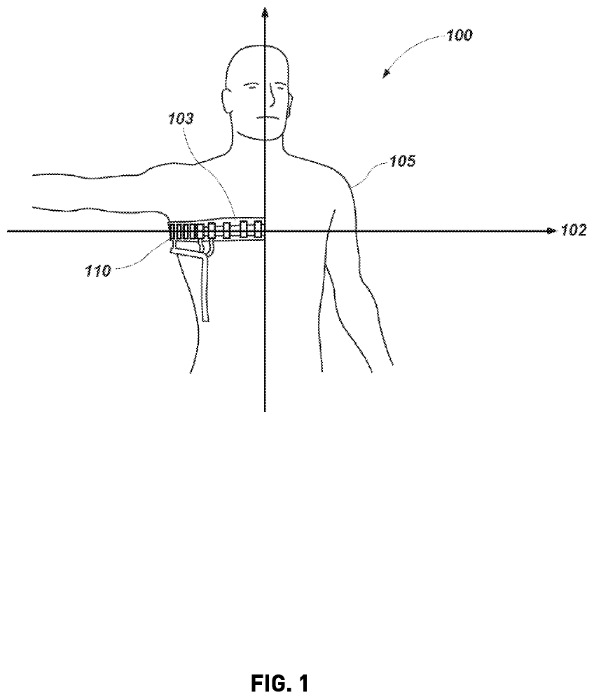

FIG. 1 is a schematic diagram of a portion of an EIT system 100 showing a plurality of electrodes 110 positioned around a region of interest (e.g., thorax) of a patient 105. The electrodes 110 of the EIT system 100 may be physically held in place by an electrode belt 103. The placement of the electrodes 110 may be transverse to the cranial caudal axis 104 of the patient and substantially parallel to axis 102. Although the electrodes 110 are shown in FIG. 1 as being placed only partially around the patient 105, electrodes 110 may be placed around the entire patient 105 depending on the specific region of interest available or desired for measurement. The electrodes 110 may be coupled to a computing system (not shown) configured to control the operation of the electrodes 110 and perform reconstruction of the EIT image.

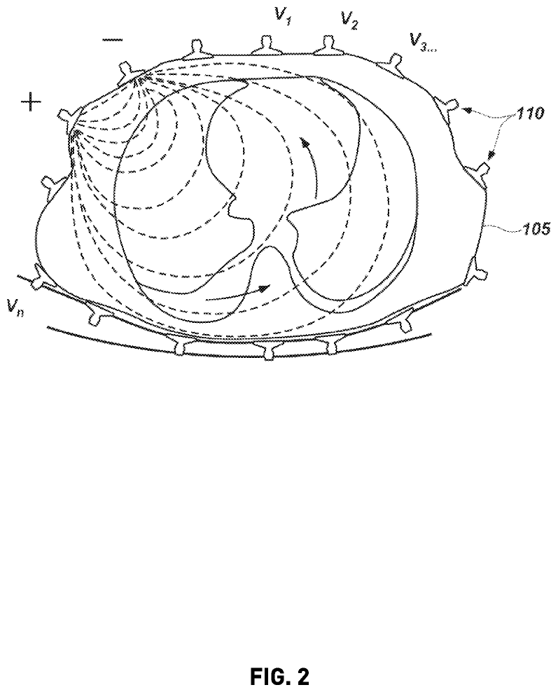

FIG. 2 is a schematic diagram showing a cross-section of the thorax of the patient 105 along a plane of the electrodes 110. A voltage may be applied to a pair of the electrodes 110 (shown by the electrodes having a + and − symbol) to inject an excitation current into the patient between the pair of the electrodes 110. As a result, voltages (e.g., V₁, V₂, V₃ . . . Vₙ) may be detected by the other electrodes 110 and measured by the EIT system 100. Current injection may be performed for a measurement cycle according to a circular pattern using different pairs of the electrodes 110 to generate the excitation current.

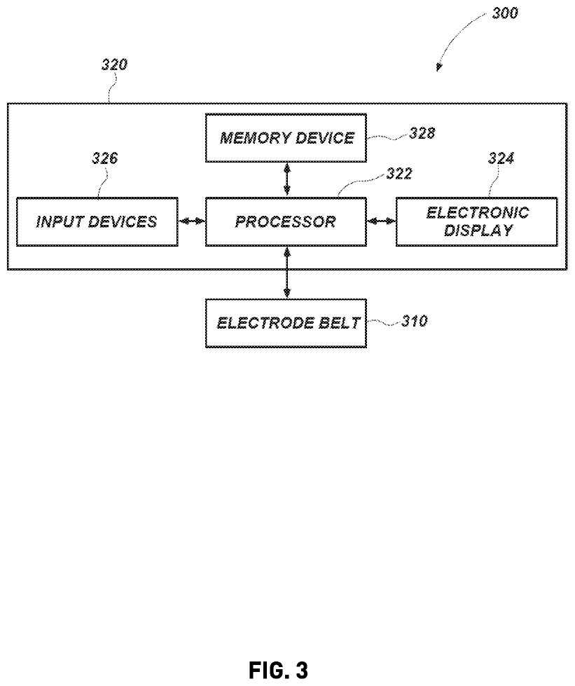

FIG. 3 is a schematic block diagram of an EIT system 300, according to an embodiment of the disclosure. The EIT system 300 may include an electrode belt 310 operably coupled with a data processing system 320. The electrode belt 310 and the data processing system 320 may be coupled together via a wired connection (e.g., cables) and/or may have communication modules to communicate wirelessly with each other. The data processing system 320 may include a processor 322 operably coupled with an electronic display 324, input devices 326, and a memory device 328. The electronic display 324 may be constructed with the data processing system 320 into a singular form factor for an EIT device coupled with the electrode belt 310. In some embodiments, the electronic display 324 and the data processing system 320 may be separate units of the EIT device coupled with the electrode belt 310. In yet other embodiments, an EIT system 300 may be integrated within another host system configured to perform additional medical measurements and/or procedures, in which the electrode belt 310 may couple to a port of the host system already having its own input devices, memory devices, and electronic display. As such, the host system may have the EIT processing software installed therein. Such software may be built into the host system prior to field use or updated after installation.

The processor 322 may coordinate the communication between the various devices as well as execute instructions stored in computer-readable media of the memory device 328 to direct current excitation, data acquisition, data analysis, and/or image reconstruction. As an example, the memory device 328 may include a library of finite element meshes used by the processor 322 to model the patient's body in the region of interest for performing image reconstruction. In some embodiments, the memory device 328 includes historical data of the impedance of a patient's lungs (such as the impedance data of a region of interest (e.g., a dependent region) and impedance data of other regions of the lungs) and/or patterns of impedance data of the patient's lungs. Input devices 326 may include devices such as a keyboard, touch screen interface, computer mouse, remote control, mobile devices, or other devices that are configured to receive information that may be used by the processor 322 to receive inputs from an operator of the EIT system 300. Thus, for a touch screen interface the electronic display 324 and the input devices 326 receiving user input may be integrated within the same device. The electronic display 324 may be configured to receive the data and output the EIT image reconstructed by the processor for the operator to view. Additional data (e.g., numeric data, graphs, trend information, and other information deemed useful for the operator) may also be generated by the processor 322 from the measured EIT data alone, or in combination with other non-EIT data according to other equipment coupled thereto. Such additional data may be displayed on the electronic display 324.

The EIT system 300 may include components that are not shown in the figures, but may also be included to facilitate communication and/or current excitation with the electrode belt 310 as would be understood by one of ordinary skill in the art, such as including one or more analog to digital converters, signal treatment circuits, demodulation circuits, power sources, etc.

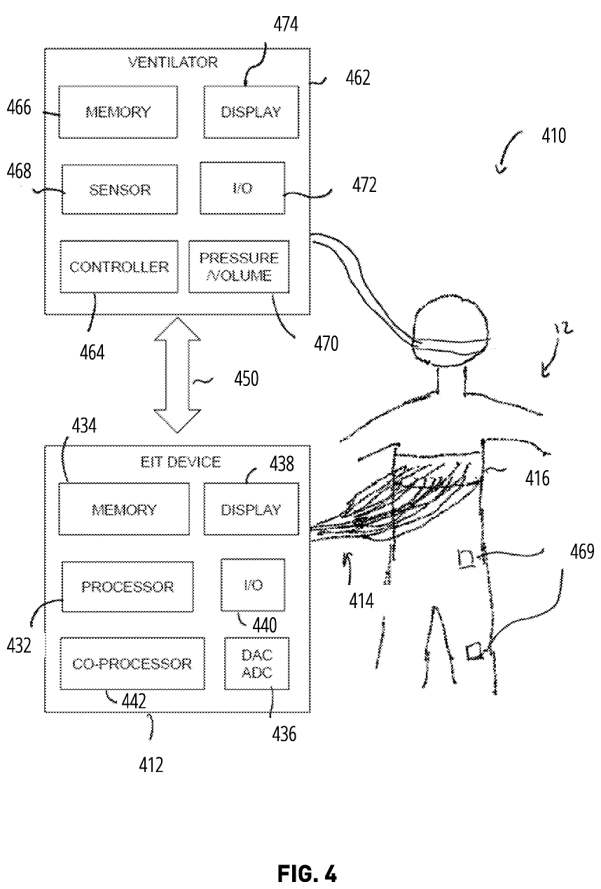

FIG. 4 provides a schematic diagram of a system 410 that includes an EIT device 412 used in conjunction with a mechanical ventilator device 462 to perform a PV maneuver. The EIT device 412 may be coupled through electrical leads 414 to an electrode belt 416 that may be placed on the patient 105. The electrode belt 416 may have one or more rows of electrodes, disposed along the perimeter of the electrode belt 416. The electrodes of the electrode belt 416 may be spatially distributed along the perimeter of the torso of patient 105. The EIT device 412 may inject electrical currents through electrodes of the electrode belt 416 and may collect the resulting electrical voltages. From the collected data, the EIT device 412 may calculate regional compliance, regional overdistension and collapse.

The EIT device 412 may perform filtering, reconstruction, and/or quantification algorithms, as detailed below. To that end, the computational device may include one or more processors 432 and one or more processing memory devices 434 (e.g., cache memory, random access memory (RAM)) to facilitate execution of the algorithms. The memory devices 434 may also include one or more protocols for performing the PV maneuver assessment.

The EIT device 412 may also include an interface module 436 configured to control the electrical currents injected into the electrical leads 414 and measure the voltages between the electrical leads 414. The interface module 436 may include, among other things, analog signal generators, analog-to-digital converters, digital-to-analog converters, digital signal processors, filters, and impedance matching circuitry, to improve signal-to-noise ratio and decrease crosstalk.

In some embodiments, the EIT device 412 may include a display 438, which may be used to provide reconstructed images, charts, and/or indices, as well as diagnostic parameters that may be calculated from the EIT images. The display 438 may also be employed to provide instructions to a practitioner performing the PV maneuver, as discussed above. In some embodiments, the display 438 may include or may be connected to a speaker or similar sound producing device to provide auditory alerts or auditory commands associated with the assessment performed during a PV maneuver.

In some embodiments, the EIT device 412 may include input/output interfaces 440 such as network interfaces, hard disk interfaces, and/or peripheral interfaces to send or receive data that may facilitate the operations of EIT, as discussed herein. For example, the EIT device 412 may be connected to the mechanical ventilator device 462 over an interface 450 that is coupled to the input/output interface 440. The interface 450 may be used to carry control commands and/or data that may be used to facilitate the assessment process, as discussed above.

In certain configurations of the system 410, such as the one illustrated in FIG. 4, the patient 105 may be connected to the mechanical ventilator device 462. The mechanical ventilator device 462 may include a controller 464 (e.g., a processor, a microcontroller) that may, in conjunction with one or more memory devices 466, control the operations of the mechanical ventilator device 462. In some embodiments, the memory devices 466 may include protocols to perform ventilator maneuvers (e.g., ventilator maneuvers), to facilitate the compliance assessment. The mechanical ventilator device 462 may include sensors 468, which may be a pressure sensor, flow sensor, or a carbon dioxide sensor. The mechanical ventilator device 462 may also include pumps 470, which may be pressure-controlled or volume-controlled pumps that provide respiratory support to the patient 105. The sensors 468 and pumps 470 may be controlled by the controller 464.

In some embodiments, the ventilator device 462 may include input/output interfaces 472 such as network interfaces, hard disk interfaces, and/or peripheral interfaces to send or receive data that may facilitate the respiratory support operations. The input/output interfaces 472 may be used to connect the mechanical ventilator device 462 to the EIT device 412 over an interface 450, as discussed above. The interface 450 may be used to carry control commands or instructions from the EIT device 412 and to provide respiratory data from sensors 468 to the EIT device 412 to facilitate the assessment, as discussed above.

In some embodiments, the ventilator device 462 may include a display 474, which may be used to provide respiratory charts, indices, and other physiological parameters associated with the respiratory support provided to the patient. In certain embodiments, the ventilator device 462 may be configured to facilitate assessments. In such devices, the display 474 may be configured to provide instructions to a practitioner performing the PV maneuver assessment, as discussed above. In some embodiments, the display 474 may include or may be connected to a speaker or similar sound producing device to provide auditory alerts or auditory commands associated with the PV maneuver.

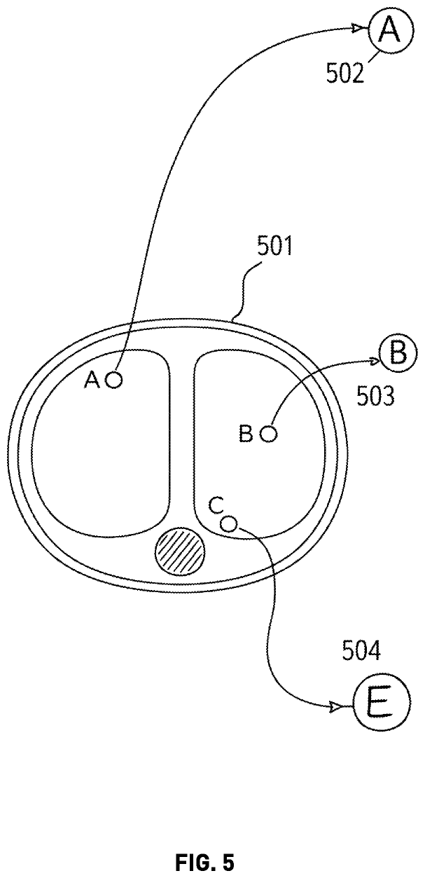

FIG. 5 illustrates a schematic representation of a transverse cross-section of a thorax 501 of a subject including distinct regions of interest (ROI). The thorax 501 includes ROI A 502, ROI B 503, and ROI C 504. These regions may be analyzed using electrical impedance tomography (EIT) data acquired during a pressure-volume (PV) maneuver. Dividing the lung into multiple regions of interest for regional compliance analysis facilitates identification of localized overdistension and collapse and identification of the pressures at which the alveoli at each region may present collapse or overdistension, rather than relying on global lung compliance measurements. The identified ROI A 502, ROI B 503, and ROI C 504 are exemplary regions of interest for the purpose of describing the method disclosed herein. In some embodiments, the regions of interest are defined based on gravitational dependence of the lung. For example, one or more regions of interest may be selected to include only non-dependent regions of the lung and one or more other regions of interest may be selected to include only dependent regions of the lung. The dependent and non-dependent regions may be defined relative to a gravitational vector and the orientation of the subject during the pressure-volume maneuver (e.g., a dependent region of the lung may refer to a region positioned relatively lower along a gravitational vector for the orientation of the subject during the pressure-volume maneuver, and a non-dependent region of the lung may refer to a region positioned relatively higher along the gravitational vector for the orientation of the subject during the pressure-volume maneuver). Thus, regional compliance, collapse, and overdistension may be assessed separately for lung regions that are entirely dependent and lung regions that are entirely non-dependent. The thorax 501 may be divided into many ROIs such as multiple ROIs having substantially equal areas, such as equal numbers of pixels in each ROI or even a different ROI for each pixel in an EIT generated image.

The PV maneuver may include slowly (e.g., at a rate within a range of from about 2 cmH₂O/s to about 5 cmH₂O/s) inflating and deflating the subject's lungs while continuously recording pressure and optionally volume data. The lungs may be inflated to a pressure within a range of from about 0 cmH₂O to about 50 cmH₂O, followed by a controlled exhalation. In some embodiments, electrical impedance tomography data used for regional compliance analysis may be acquired during a deflation limb of the pressure-volume maneuver, where the lungs are deflated from a pressure greater than about 30 cmH₂O to a pressure lower than about 15 cmH₂O. Regional impedance changes may be concurrently monitored using EIT. The inflation (i.e., inspiratory) phase may be executed over a period of from about 10 to about 50 seconds to ensure a controlled and uniform pressure increase, while the deflation (i.e., expiratory) phase may be executed over a period of within a range of from about 20 to about 40 seconds. In some embodiments, information from the inspiratory limb (e.g., phase) of the pressure-volume maneuver is not used to assess regional compliance. In some embodiments, regional compliance is determined during the pressure-volume maneuver in an absence of a breathing cycle. For example, the pressure-volume maneuver may be performed as a quasi-static maneuver in which ongoing tidal inhalation and exhalation cycles are absent while pressure and impedance data used for the regional compliance determination are acquired. Thus, the determined regional compliance may reflect regional lung mechanics during the pressure-volume maneuver rather than compliance derived from a breathing cycle.

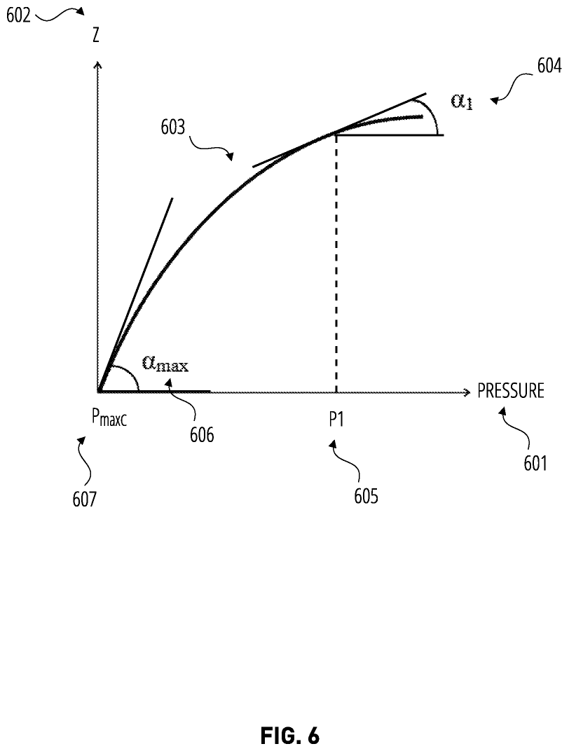

FIG. 6 is a graph illustrating an expiratory limb of a PV curve 603 for ROI A 502. Pressure 601 is plotted on the x-axis of the graph and impedance (z) 602 is plotted on the y-axis. Angle α₁ 604 represents the angle formed by the tangent of the PV curve 603 of the PV curve at pressure P₁ 605 with respect to the x-axis. Angle αₘₐₓ 606 represents the angle of the maximum tangent to the curve, which occurs at pressure P_{maxC} 607.

A tangent observed at the expiratory limb of a PV curve at pressure P₁ 605 forms the angle α₁ 604, which graphically represents the local slope of the PV curve and therefore the local compliance at pressure P₁ 605 in ROI A 502. Maximum regional compliance is observed at P_{maxC} 607, where the local slope of the PV curve, graphically represented by tangent angle αₘₓ 606, is greatest. In any given ROI (e.g., ROI A 502, ROI B 503, ROI C 504) there may be multiple alveoli. At PmaxC 607, regional compliance in ROI A 502 is at or near a maximum. At pressures outside of the P_{maxC} 607 alveoli compliance may decrease due to collapse or overdistension. For example, at pressures higher than P_{maxC} 607 compliance decreases, as indicated by the reduction in the tangent angle (α₁ 604<αₘₐₓ 606). Thus, alveoli in the ROI A 502 begin to become overdistended at pressures higher than P_{maxC} 607. Accordingly, by determining the local slope, or the corresponding tangent angle, at various pressure levels along the curve, a pressure level P_{maxC} may be identified at which regional compliance within a given region of interest is maximized.

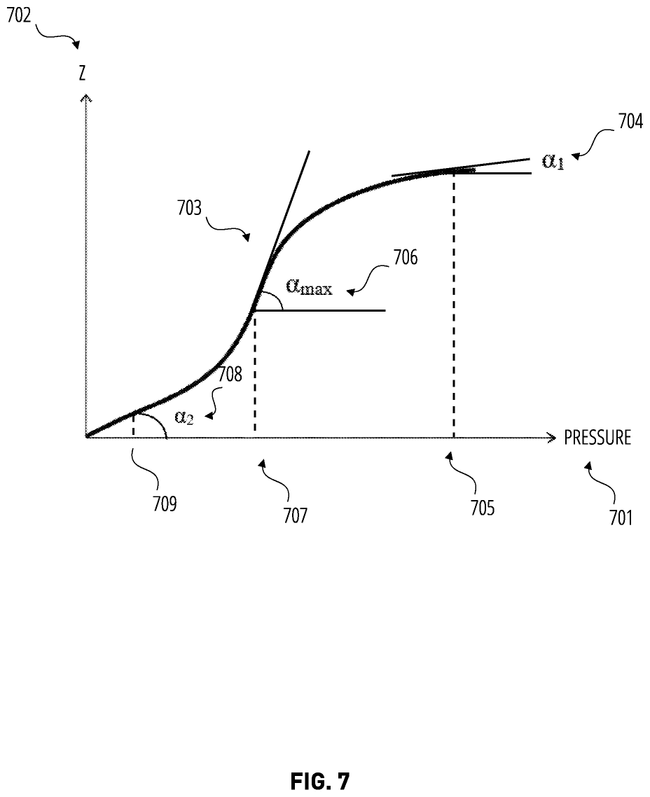

FIG. 7 is a graph illustrating an expiratory limb of PV curve 703 for ROI B 503. Pressure 701 is plotted on the x-axis of the graph and impedance (z) 702 is plotted on the y-axis. The curve describes the relationship between airway pressure and impedance-derived lung volume during the expiratory phase of a PV maneuver in ROI B. Tangents are applied to the curve at multiple pressures to determine local compliance values.

At P_{maxC} 707, the tangent angle αmax 706 graphically represents the greatest local slope of the PV curve 703 and therefore the maximum regional compliance. As discussed above, at pressures outside of the P_{maxC} 707 compliance of some of the alveoli may decrease, due to collapse or overdistension. For example, at a higher pressure P₁ 705, the tangent angle α₁ 704 is smaller than αₘₐₓ 706, indicating reduced regional compliance consistent with overdistension in ROI B 503. At a lower pressure P₂ 709, the tangent angle α₂ 708 is also smaller than αₘₐₓ 706, indicating reduced regional compliance consistent with collapse in ROI B 503. This pattern suggests that ROI B 503 is susceptible to overdistension at pressures higher than P_{maxC} 707, and to collapse at pressures below P_{maxC} 707. Accordingly, within each ROI opposite behaviors can be observed according to the pressure applied during a PV maneuver.

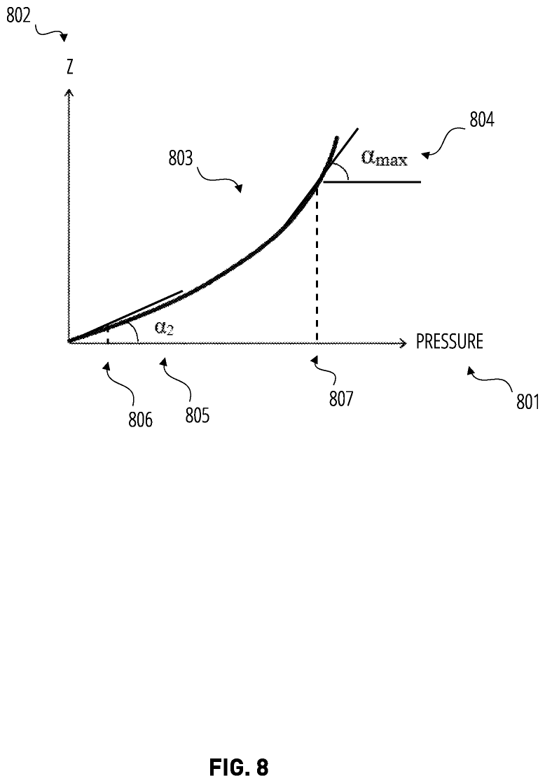

FIG. 8 illustrates the expiratory limb of the PV curve 803 for ROI C 504. Pressure 801 is plotted on the x-axis of the graph and impedance (z) 802 is plotted on the y-axis. At P_{maxC} 807, tangent angle αₘₐₓ 804 graphically represents the greatest local slope of the PV curve 803 and therefore the maximum regional compliance in ROI C 504. At a lower pressure P₂ 806, the measured tangent angle α₂ 805 is smaller than αₘₐₓ 804, indicating reduced compliance. This indicates reduced regional compliance at lower pressures, consistent with collapse in ROI C 504. Unlike ROI B 503, which experiences both overdistension and collapse, and ROI A 502, which experiences overdistension, ROI C 504 primarily demonstrates the occurrence of collapse at pressures below P_{maxC} 807, with no indication of overdistension at higher pressures.

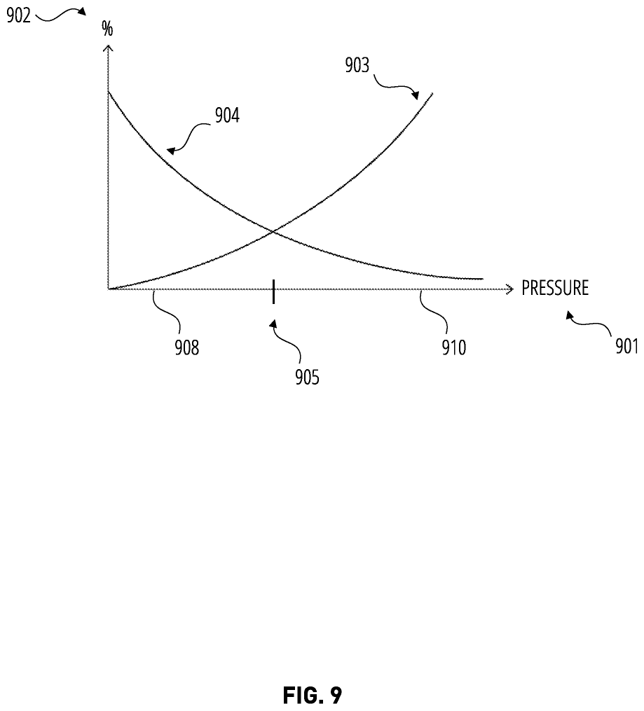

The P_{maxC} 807 of the ROI C 504 illustrated in FIG. 8 is a different pressure than the P_{maxC} 707 of the ROI B 503 illustrated in FIG. 7 and each of the P_{maxC} 807 and the P_{maxC} 707 are a different pressure from the P_{maxC} 607 of the ROI A 502 illustrated in FIG. 6. Thus, at each ROI the associated Pₘₐₓ will potentially be different. After the Pₘₐₓ for each ROI is found, cumulative collapse or overdistension of the entire lung or several ROIs may be estimated at each pressure based on the measurements taken in each ROI. This may be used to find a pressure at which there is the best compromise between cumulative collapse and overdistension (e.g., at the intersection 905 of the graph at FIG. 9, discussed below).

FIG. 9 is a graph illustrating the relationship between cumulative overdistension 903 and cumulative collapse 904 as a function of pressure in the regions of interest analyzed, in this case ROI A 502, ROI B 503, and ROI C 504. Pressure 901 is plotted on the x-axis of the graph and % overdistended or collapsed 902, respectively, is plotted on the y-axis. Pressure (P) levels 905, 908, and 910, discussed in more detail below, are indicated on the x-axis.

The percentage collapse in each ROI, and the cumulative collapse may be calculated by first calculating compliance in the ROI at a number (n) pressures during a PV maneuver, where n=2 or more. After calculating the compliance in the ROI, the percent change in compliance at a certain pressure of the PV maneuver is determined for each pixel in relation to its “best compliance”, according to the formula:

$${Collapse}_{ROI} ( \% ) = \frac{( Best {compliance}_{ROI} - Current {Compliance}_{ROI} ) \times 100}{Best {compliance}_{ROI}}$$

A Best Compliance_{ROI} may be determined to be the highest compliance value for each ROI across all pressures of the PV curve at which the ROI compliance was measured, and Collapse_{ROI} (%) is set to 0 if the Best Compliance Roi has not yet been achieved for that ROI. After determining the percent change in compliance, the cumulative collapse for the aggregate of the ROIs analyzed (from ROI₁ to ROIₙ) is estimated at each pressure of the PV curve at which the ROI compliance was measured as the weighted average of Collapse_{ROI}, where the weighting factor is the best ROI compliance, which is assumed to indicate the functional size of the lung compartment represented by a certain ROI:

$$Cumulated collapse ( \% ) = \frac{{\sum }_{ROI = 1}^{n} ( {Collapse}_{ROI} ( \% ) \times Best {compliance}_{ROI} )}{{\sum }_{ROI = 1}^{n} ( Best {compliance}_{ROI} )}$$

During the expiratory phase of a PV maneuver, the high initial pressure levels may lead to lung overdistension, which can be assessed as a percent decrease in ROI compliance in relation to its peak value (Best Compliance_{ROI}) measured at lower pressures of the PV maneuver.

These changes in ROI compliance may be weight-averaged according to each ROI's maximal compliance to compensate for different amounts of alveolar units contained within each ROI. Cumulative alveolar overdistension may be estimated according to the formula:

$$Cumulated Overdistension ( \% ) = \frac{{\sum }_{ROI = 1}^{n} ( {Overdistension}_{ROI} \times Best {compliance}_{ROI} )}{{\sum }_{ROI = 1}^{n} ( Best {compliance}_{ROI} )}$$

where overdistension_{ROI} (%) is set to 0 if the Best Compliance_{ROI} has already been achieved for that ROI.

In some embodiments, cumulative overdistension and cumulative collapse at each pressure of the pressures at which regional compliance is determined may be computed using region-specific weights based on compliance change relative to peak compliance. For each region of interest, the system may determine whether the pressure is above or below P_{maxC} and may determine a weight based on a percent change in compliance of the region of interest at the pressure relative to the compliance of the region of interest at P_{maxC}. The system may estimate cumulative overdistension as a weighted average of overdistension values for regions of interest for which the pressure is above P_{maxC} and may estimate cumulative collapse as a weighted average of collapse values for regions of interest for which the pressure is below P_{maxC}. In some embodiments, the weight may be the percent change in compliance, may be proportional to the percent change in compliance, or may be otherwise derived from the percent change in compliance.

As a particular example, with reference to FIG. 7, at pressures above P_{maxC} 707, ROI B 503 experiences overdistension, and at pressures below P_{maxC} 707, ROI B 503 experiences collapse. By determining a P_{maxC} for each region of interest, cumulative overdistension and cumulative collapse may be determined and a graphical representation may be output.

As can be seen in FIG. 9, cumulative overdistension 903 increases at higher pressures, indicating that lung regions become overdistended and lose compliance. Conversely, cumulative collapse 904 increases at lower pressures, signifying that lung regions collapse due to insufficient airway pressure. At pressure P_{med} 905, the cumulative overdistension 903 and cumulative collapse 904 curves intersect. P_{med} 905 thus represents a pressure level, or range of pressure levels, with the best compromise between cumulative lung collapse and overdistension.

A system according to embodiments of the disclosure may process pressure-dependent compliance data across the multiple regions of interest to generate and display the graphical representation shown in FIG. 9. The system may compute and output a marker corresponding to the pressure P_{med} 905, which represents a balance point between cumulative overdistension and cumulative collapse. The system may generate a line corresponding to P_{med} 905 on the graphical representation to visually indicate a preferred pressure level or pressure range. The system may also identify and display a recommended pressure range to an operator, based on the intersection of the cumulative overdistension and cumulative collapse curves, thereby providing guidance for setting PEEP levels.

In some embodiments, a system in accordance with embodiments of the disclosure is further configured to automatically control the mechanical ventilator to apply a PEEP level at or near the determined P_{med} 905, or within a threshold range identified from the data. The system may also issue an alert if the calculated P_{med} 905 falls outside a predefined safe range, or if significant shifts in P_{med} 905 are detected over time. The system may store historical pressure-compliance data and perform comparisons to detect trends in regional lung compliance, facilitating assessment of progression or improvement in ventilation.

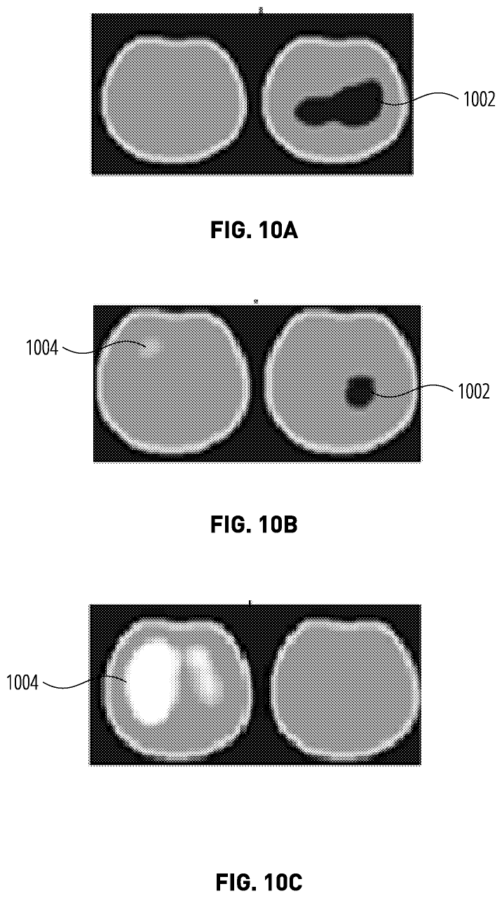

FIGS. 10A-10C depict EIT images of lungs taken at various pressure levels showing collapsed regions 1002 and overdistended regions 1004.

FIG. 10A is an EIT image of lungs taken at P 908 of FIG. 9. At P 908 the lungs display a collapsed region 1002 without any overdistended regions 1004, indicating that cumulative collapse 904 is significantly greater than cumulative overdistension 903 at this pressure level.

FIG. 10B is an EIT image of lungs taken at P_{med} 905. At P_{med} 905 the lungs display a relatively low amount of collapsed regions 1002 and overdistended regions 1004, indicating that cumulative collapse 904 is well balanced with cumulative overdistension 903 at this pressure level.

FIG. 10C is an EIT image of lungs taken at P 910 of FIG. 9. At P 910 the lungs display overdistended regions 1004 without any collapsed regions 1002, indicating that cumulative overdistension 903 is significantly greater than cumulative collapse 904 at this pressure level.

### Examples

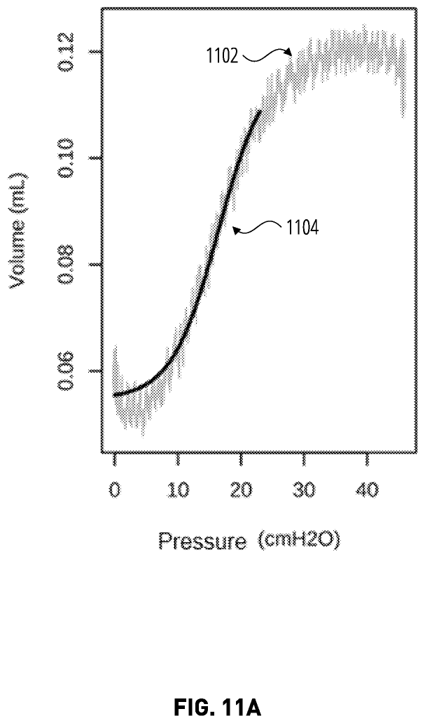

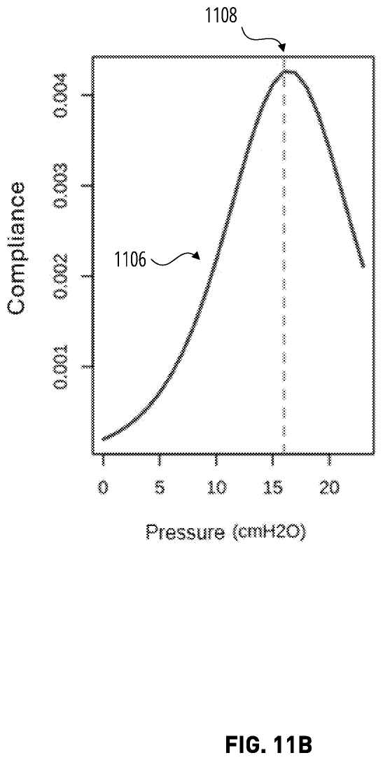

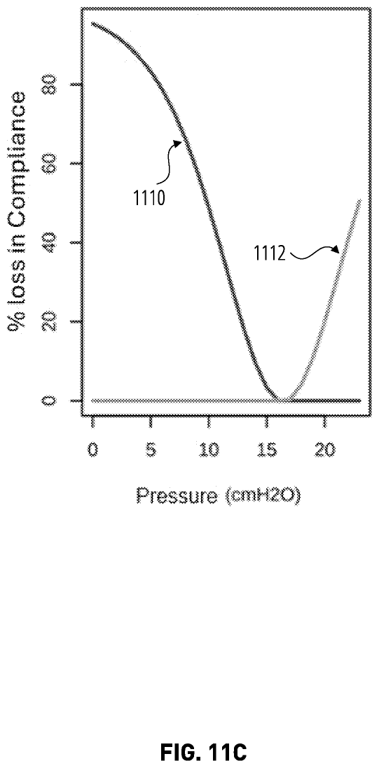

FIG. 11A displays raw EIT data 1102, which was measured at the ROI C during the expiratory phase of a PV maneuver. The data was then fitted to create a modeled curve 1104.

FIG. 11B is a graph illustrating a curve representing local slopes, graphically represented by tangents, of the modeled curve 1106 of FIG. 11A fitted to the impedance data, with compliance plotted on the y-axis and pressure plotted on the x-axis. The slope was calculated as the derivative of Volume with respect to Pressure (dVolume/dPressure) or (dZ/dP). An inflection point 1108 of the curve is noted at a P_{maxC} of 16 cmH₂O. It was observed that compliance increased from about 0 to about 0.004 in a sigmoidal fashion from a pressure of about 0 cmH₂O to about 16 cmH₂O, and decreased above 16 cmH₂O.

FIG. 11C is a graph depicting the collapse 1110 and overdistension 1112 observed in ROI C. Overdistension refers to the percentage loss of compliance in that region for pressures above P_{maxC}, while Collapse indicates the percentage loss of compliance for pressures below P_{maxC}. The overdistension curve increased from 0 to about 50% at a pressure of about 17 cmH2O, while collapse exhibited a decreasing sigmoidal trend, decreasing from about 90% at a pressure of about 0 cmH2O to about 0% at a pressure of about 16 cmH2O.

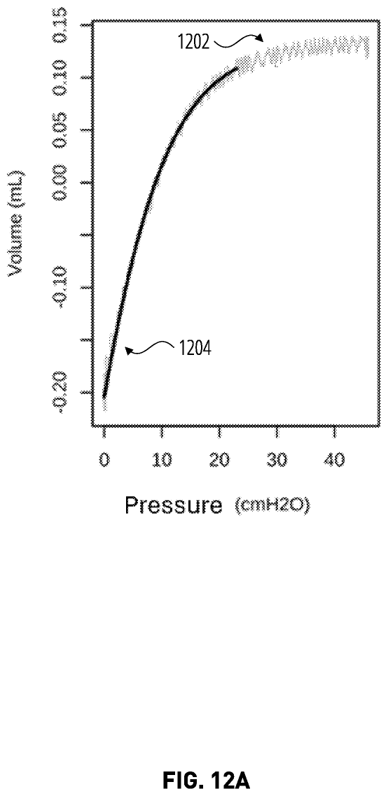

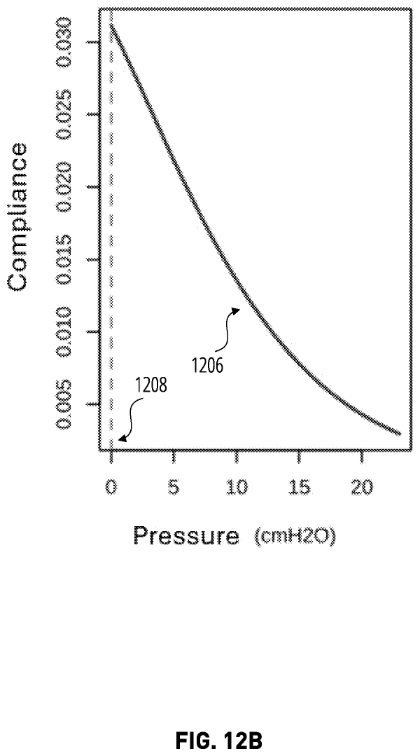

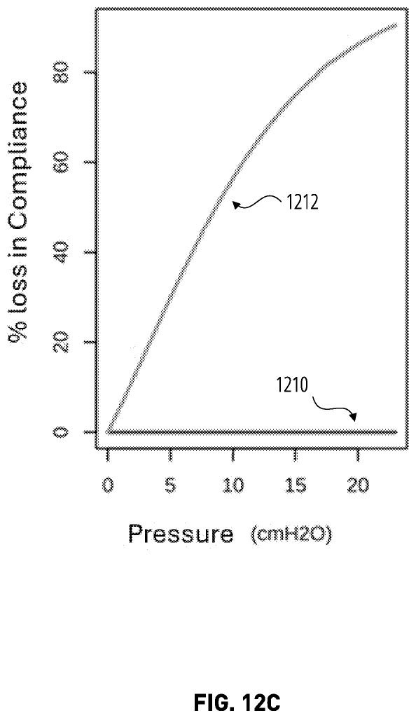

FIG. 12A displays raw EIT data 1202 measured at the ROI A during the expiratory phase of a Pressure-Volume (PV) maneuver. The raw impedance data was fitted to create a modeled curve 1204.

FIG. 12B illustrates a curve representing the local slopes, graphically represented by tangents, of the modeled curve 1206 fitted to the impedance data of FIG. 12A. The slope was calculated as the derivative of Volume with respect to Pressure (dVolume/dPressure) or (dZ/dP). In this instance, regional compliance improved as pressure decreased. The highest compliance was noted at the lowest pressure 1208 of the Pressure-Volume (PV) maneuver. However, no inflection point was detected within the range of pressures applied during the maneuver. This curve suggests a relief of overdistension during the expiratory phase of the PV maneuver, with no collapse observed at any pressure.

FIG. 12C is a graph depicting the collapse 1210 and overdistension 1212 observed in ROI A. Overdistension refers to the percentage loss of compliance in that region for pressures above P_{maxC}. Since P_{maxC} in this case was the lowest pressure applied during the PV maneuver, no collapse was observed, while overdistension increased in a sigmoidal fashion as pressure increased.

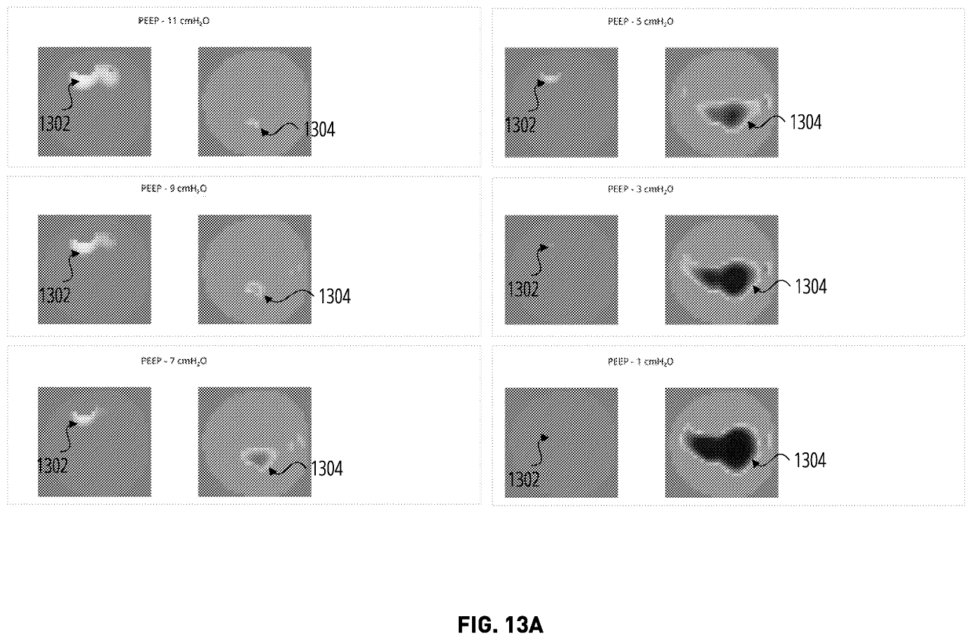

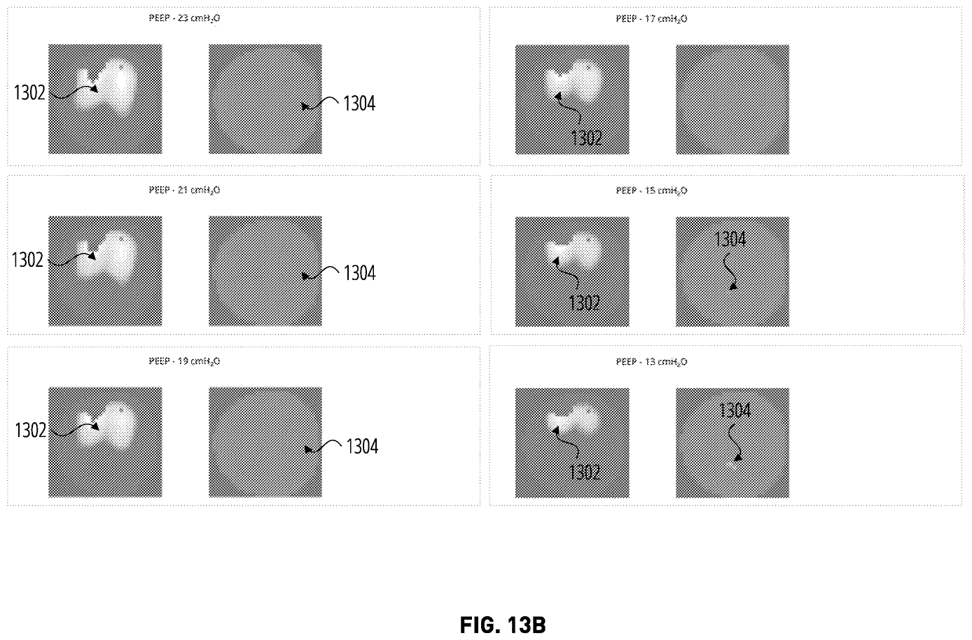

FIGS. 13A and 13B provide graphical representations of regional overdistension 1302 and collapse 1304 for Regions of Interest (ROIs) C and A, respectively, at various pressure levels during a Pressure-Volume (PV) maneuver. In ROI C (FIG. 13A), collapse 1304 increased as pressure decreased, while overdistension 1302 decreased as pressure decreased. In ROI A (FIG. 13B), collapse 1304 stayed flat at about 0% as pressure decreased, while overdistension 1302 decreased as pressure decreased.

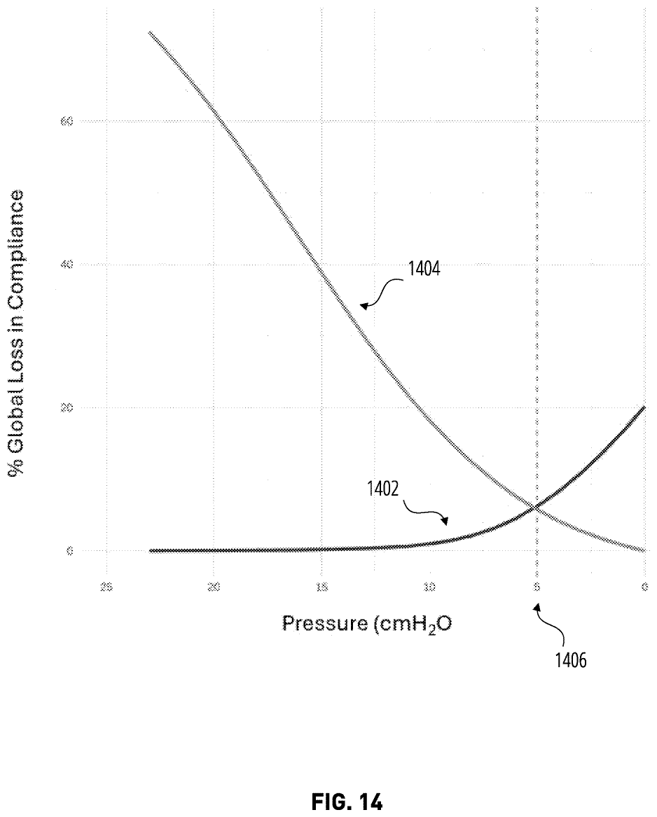

FIG. 14 is a graph illustrating the cumulative collapse 1402 (e.g., a loss of compliance as pressure is lowered) and overdistension 1404 (e.g., a total gain in compliance when pressure is lowered) for the combined Regions of Interest (ROIs) A and C. Cumulative collapse 1402 decreased as pressure increased, while cumulative overdistension 1404 increased as pressure increased. An ideal PEEP level 1406 was found to be at a pressure of about 5 cmH₂O, indicated with a dashed line.

By analyzing the tangent to the expiratory limb of the PV curve and outputting graphical representations of the regional and cumulative overdistension and collapse, regional variations in compliance may be taken into account by operators during selection of PEEP levels for mechanical ventilation. The methods and systems according to embodiments of the disclosure thus facilitate PEEP titration in a regional manner over a short period of time, minimizing the risk of lung injury and hemodynamic impairment induced by high pressures compared to conventional systems and methods. In various implementations, the system may provide a graphical output, visual pressure recommendations, automated ventilator control, or combinations thereof, based on pressure-response analysis across multiple lung regions.

In further embodiments, the regional compliance assessment described above may be implemented using a quasi-static PV maneuver and a distending-pressure input representative of alveolar pressure. In these embodiments, the same underlying regional mechanics may be characterized using regional compliance curves Cᵢ(P), from which lower-pressure compliance loss and higher-pressure compliance loss may be derived as respective indicators of derecruitment/collapse and overdistension. Thus, the methods and systems described below may be used in place of, or in combination with, the P_{maxC}-based framework described above to identify a pressure associated with a compromise between collapse and overdistension.

Accordingly, further embodiments of the disclosure are directed to implementations that utilize quasi-static PV maneuvers (e.g., slow inflation and/or deflation ramps performed with low, substantially constant flow and, optionally, brief end-inspiratory and/or end-expiratory pauses such that airflow is near zero and resistive and inertial pressure components are small relative to elastic pressure) and explicit estimation of alveolar (e.g., distending) pressure for regional compliance analysis. These embodiments may be implemented in combination with the systems and methods described above for deriving regional compliance, cumulative collapse, and cumulative overdistension from pressure-impedance curves. The following description provides additional examples of how a system according to embodiments of the disclosure may estimate alveolar pressure and regional compliance and compute related indices using the PV maneuver.

A system according to embodiments of the disclosure may be configured to determine alveolar pressure Pₐₗᵥ(t) from proximal airway pressure P_{aw}(t) for use as a pressure input to regional compliance analysis. The system may be in communication with a ventilator or pneumotachograph configured to provide a time-series of proximal pressure P_{aw}(t) (e.g., pressure at the airway opening) and a time-series of airflow \dot{V}(t). The system may be configured to store or receive a total system resistance R_{sys} that includes the patient's airway resistance and a resistance of the endotracheal tube and breathing circuit. During a respiratory maneuver, the total pressure at the airway opening P_{aw}(t) overcomes both an elastic component (e.g., lung and chest wall recoil) and a resistive component due to airflow through the conducting airways and artificial airway. Neglecting inertial terms, the elastic distending pressure at the alveoli may be estimated by applying a resistive correction to the proximal signal according to a relationship of the form:

$${P}_{alv} ( t ) \approx {P}_{aw} ( t ) - \dot{V} ( t ) \cdot {R}_{sys} .$$

In some embodiments, R_{sys} is obtained from ventilatory parameters measured before or after the PV maneuver, such as resistance measured by a ventilator or patient monitor during standard breaths. These parameters may be measured by the system, input by a user through a user interface, or received electronically from a mechanical ventilator or patient monitor. For quasi-static PV maneuvers in which flow is maintained very low or is zero, the term \dot{V}(t)·R_{sys} is small and may be treated as a substantially constant offset. In such cases, P_{aw}(t) may be used as a surrogate for Pₐₗᵥ(t), but the system may still apply the resistive correction to derive a pre-processed pressure input vector Pₐₗᵥ(t) that removes the offset due to tube and airway resistance. The regional compliance estimation described in the present disclosure may use this pre-processed Pₐₗᵥ(t) as the pressure axis so that the calculated regional compliance Cᵢ(P) reflects elastic mechanical properties of the lung parenchyma.

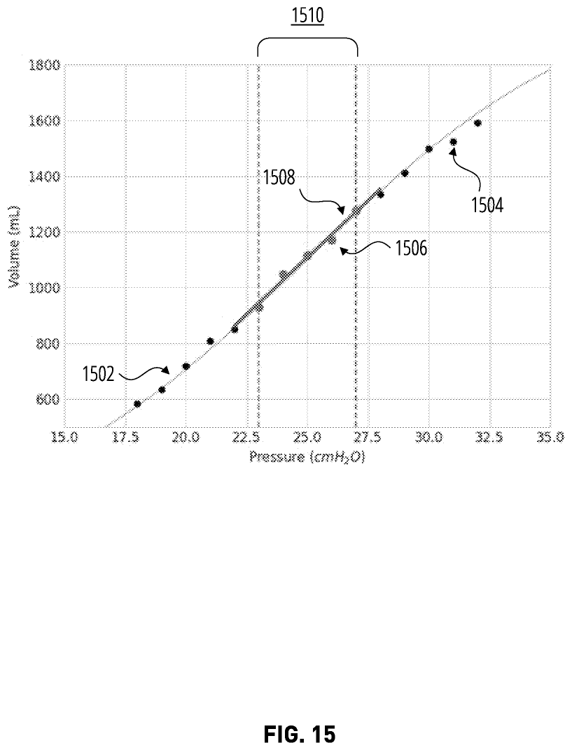

With reference to FIG. 15, the system may be configured to estimate regional compliance profiles using a local sliding-window regression approach that approximates the derivative dV/dP or dZ/dP for each compartment. FIG. 15 illustrates a curve 1502 and discrete data samples 1504. An EIT system may provide time-series data for each compartment i in the form of regional impedance variation ΔZᵢ(t), which is treated as linearly related to regional volume Vᵢ(t). Because the compliance-loss analysis may be based on relative changes normalized to a peak compliance or a best-compliance value, impedance variation may serve as a surrogate for regional volume while preserving validity of the analysis. The system may be configured to receive or compute a synchronized distending pressure signal, such as Pₐₗᵥ(t) derived as described above. The lung may be modeled as a set of parallel compartments corresponding to EIT pixels or regions of interest, with total volume equal to the sum of the regional volumes Vᵢ(t). As illustrated in FIG. 15, the system may define a pressure window 1510 centered on a target pressure and may estimate a local slope 1508 from data samples 1506 within that pressure window 1510, the local slope corresponding to a local regional compliance for the compartment.

In some embodiments, the system defines a set of target pressures Pₖ that span the range of pressures encountered during the decremental (e.g., deflation) limb of the PV maneuver. For each target pressure Pₖ the system may define a pressure window of width ΔP (for example, about 2 cmH₂O) centered on Pₖ. The system may select all time samples tₖ within the deflation limb such that Pₐₗᵥ(tₖ) lies within the interval [Pₖ−ΔP/2, Pₖ+ΔP/2]. FIG. 15 illustrates an example in which samples within the pressure window are used to estimate a local regression line. The particular window width shown in FIG. 15 of about 4 cmH₂O is exemplary only, and other window widths may be used. For the i-th compartment, the system may assume a local linear relationship within this window:

$${V}_{i} ( {t}_{k} ) \approx {a}_{i} ( {P}_{k} ) + {C}_{i} ( {P}_{k} ) \cdot {P}_{alv} ( {t}_{k} )$$

where Cᵢ(Pₖ) represents a local regional compliance at pressure Pₖ and aᵢ(Pₖ) is an intercept term. The system is configured to estimate Cᵢ(Pₖ) via ordinary least squares regression over the selected samples, minimizing a sum of squared residuals between measured Vᵢ(tₖ) and modeled values. A slope of the local regression line may correspond to Cᵢ(Pₖ). In some embodiments, Vᵢ(t) is implemented directly as impedance variation ΔZᵢ(t) used as a surrogate for volume.

In some embodiments, signal filtering is applied before regression to mitigate non-ventilation-related phenomena such as cardiogenic oscillations, which manifest as high-frequency perturbations superimposed on the ventilation signal. The system may apply temporal filtering and/or pressure-domain filtering (e.g., sorting samples by pressure and smoothing) to reduce oscillations while preserving the quasi-static PV relationship. In some embodiments, pressure-domain filtering may be used in place of, or in addition to, low-pass temporal filtering because temporal filtering may introduce phase lag, whereas pressure-domain filtering may reduce cardiac-related perturbations while preserving static mechanical characteristics of the PV loop. By repeating the sliding-window regression for multiple Pₖ values across the deflation limb, the system generates a regional compliance curve Cᵢ(P) for each compartment.

In some embodiments, the system is configured to estimate regional compliance by fitting a global analytical model to pressure-volume or pressure-impedance data for each compartment and differentiating the fitted model. The system may assume that the static PV curve follows a sigmoidal shape described by a Venegas-type equation. For a given compartment i, the system may model volume Vᵢ(P) as:

$${V}_{i} ( P ) = a + \frac{b}{1 + {e}^{- ( P - c ) / d}}$$

where a is the lower asymptote (i.e., volume at zero pressure), b is vital capacity of the compartment (i.e., total volume change), c is the inflection point pressure (i.e., pressure at highest compliance), and d is a parameter related to the width of the linear segment.

The system is configured to estimate the parameters for each compartment i using non-linear least squares optimization, fitting Vᵢ(P) (or an impedance-derived surrogate) to the measured pressure-volume or pressure-impedance data along the deflation limb. Once parameters are obtained, the system computes an analytical regional compliance function Cᵢ(P) by differentiating Vᵢ(P) with respect to pressure P. This produces a smooth, continuous compliance curve Cᵢ(P) for each lung region and provides analytical parameters that describe the mechanical behavior of that region.

In some embodiments, the system is configured to characterize the functional status of lung tissue by calculating compliance loss indices from the regional compliance curves Cᵢ(P). For each compartment i, the system identifies a maximum (peak) compliance Cₚ, and the pressure at which it occurs, P_{peak,i}. Compliance loss Lᵢ at a given pressure P is quantified as:

$${L}_{i} ( P ) = {C}_{peak , i} - {C}_{i} ( P )$$

In some embodiments, Lᵢ(P) is classified into Low Pressure Compliance Loss (LPCL) and High Pressure Compliance Loss (HPCL) according to the relation between P and P_{peak,i}. For pressures P lower than P_{peak,i}, Cᵢ(P) is on the rising portion of the compliance curve and Lᵢ(P) corresponds to LPCL, associated with derecruitment. For pressures P higher than P_{peak,i}, Cᵢ(P) is on the descending portion of the curve and Lᵢ(P) corresponds to HPCL, associated with overdistension. In some embodiments, the pressure Ppeak,i at which Cᵢ(P) is maximal corresponds to the pressure identified above as PmaxC for a given region or compartment. Likewise, LPCL may correspond to lower-pressure compliance reduction associated with collapse or derecruitment, and HPCL may correspond to higher-pressure compliance reduction associated with overdistension. Accordingly, aggregated LPCL and HPCL indices may be used to generate pressure-dependent curves analogous to the cumulative collapse and cumulative overdistension curves described above.

In some embodiments, the system is configured to compute global LPCL(P) and HPCL(P) indices by aggregating regional losses weighted by peak compliance. LPCL(P) may be given by a weighted sum or average of Lᵢ(P) over compartments with P<P_{peak,i}, with weights proportional to C_{peak,i}, and HPCL(P) may be given by a corresponding aggregation over compartments with P>P_{peak,i}. The system may present LPCL(P) and HPCL(P) as continuous curves against pressure. A user interface may be configured to indicate an intersection of the LPCL and HPCL curves as a pressure of interest, representing a compromise between derecruitment and overdistension, in analogy with the method and system described above with reference to FIGS. 1-14.

In some embodiments, the system is configured to generate functional images of LPCL and HPCL at each pressure level. Using the values Lᵢ(P) and the classification based on P_{peak,i}, the system may construct electrical impedance tomography tomograms in which compartments exhibiting HPCL and LPCL are spatially mapped and visually distinguished. The system may compute and display percentages of lung area affected by HPCL and LPCL at each pressure, enabling assessment of spatial patterns during a decremental pressure maneuver. In some embodiments, the functional images may be used to evaluate physiologic consistency of the measurements, including coherence with an expected influence of a gravitational vector on lung mechanics.

In some embodiments, the validity of regional compliance estimation relies on quality of the input data and maintenance of quasi-static conditions throughout the PV maneuver. The system according to embodiments of the disclosure may be configured to implement pre-processing steps to detect and manage artifacts. The system may monitor airway pressure Pₐw(t), airflow \dot{V}(t), and regional impedance signals for signatures of patient-ventilator interaction, including coughing, swallowing, and active inspiratory efforts, which manifest as sharp, non-physiological transients and disrupt the monotonic deflation required for analysis. The system may also monitor for patient movement, which may shift the EIT electrode plane relative to lung tissue or alter thoracic geometry, and for EIT signal noise due to poor electrode contact, lead detachment, or electromagnetic interference, which may introduce high-amplitude noise or baseline drift.

In some embodiments, the system is configured to detect these events based on changes in Pₐw(t), \dot{V}(t), and impedance signals and to flag or exclude corresponding data segments from analysis. The system may apply pressure-domain filtering by sorting samples by pressure and smoothing along the pressure axis to reduce cardiogenic oscillations while preserving the static mechanical characteristics of the PV loop. Implementing these pre-processing and artifact-rejection steps ensures that compliance and compliance-loss indices reflect regional lung mechanics rather than transient artifacts.

In some embodiments, the system is configured to compensate for the influence of PV maneuver decay rate on regional compliance estimation. The decay rate is the rate at which pressure is decreased during the deflation limb of the PV maneuver and may be within a range of from about 2 cmH₂O/s to about 10 cmH₂O/s. The assessment of regional lung compliance at the expiration limb may be influenced by flow and resistance as a function of this decay rate, because higher decay rates generate higher flows and therefore larger resistive pressure drops.

In some embodiments, the system is configured to apply a correction term to account for the combined effect of \dot{V} and Rsys at different decay rates. The correction may be linear, quadratic, based on a fixed value, or based on patient-specific lung mechanics such as resistance or compliance measured at the airway when the PV maneuver is not being executed. These measurements may be performed by the device itself or obtained through integration with external devices such as a mechanical ventilator. In some embodiments, the system is configured to compare the crossing point between LPCL and HPCL obtained using a stepwise PEEP titration method with the crossing point obtained from regional compliance assessment on the expiration limb of a PV maneuver. The system may apply one or more correction strategies for flow and resistance such that, particularly at higher decay rates, the PV-based crossing point approaches the stepwise-based crossing point, with higher decay rates requiring higher correction magnitudes.

While embodiments of the disclosure may be susceptible to various modifications and alternative forms, specific embodiments have been shown by way of example in the drawings and have been described in detail herein. However, it should be understood that the disclosure is not limited to the particular forms disclosed. Rather, the disclosure encompasses all modifications, variations, combinations, and alternatives falling within the scope of the disclosure as defined by the following appended claims and their legal equivalents.

What is claimed is:

1. A method for assessing regional lung compliance in a mechanically ventilated patient, the method comprising:
acquiring electrical impedance tomography data representative of a plurality of lung regions while a pressure-volume maneuver is performed on the mechanically ventilated patient;
associating pressure values from the pressure-volume maneuver with corresponding regional electrical impedance tomography data to obtain a regional pressure-impedance curve;
determining the regional compliance at more than two different pressures;
determining a pressure P_{maxC} at which the regional compliance is greatest;
determining an occurrence of at least one of overdistension at pressures above P_{maxC} and collapse at pressures below P_{maxC};
calculating cumulative overdistension and cumulative collapse based on the occurrence of overdistension and collapse at the plurality of lung regions at each of the more than two different pressures; and
outputting a pressure P_{med} at which curves representing the cumulative overdistension and the cumulative collapse over the more than two different pressures intersect.

2. The method of claim 1, wherein determining the regional compliance at more than two different pressures comprises determining a slope of the regional pressure-impedance curve at each of the more than two different pressures.

3. The method of claim 2, wherein determining the slope of the regional pressure-impedance curve comprises calculating an angle of a tangent to the regional pressure-impedance curve.

4. The method of claim 1, wherein determining the regional compliance at more than two different pressures comprises:
defining a set of target pressures Pₖ spanning a range of pressures encountered during a deflation limb of the pressure-volume maneuver;
defining a pressure window of width ΔP centered on each target pressure Pₖ;
selecting time samples tₖ within the deflation limb such that Pₖ(tₖ) lies within an interval
$$[ {P}_{k} - Δ P / 2 , {P}_{k} + Δ P / 2 ] ;$$
estimating Cᵢ(Pₖ) by ordinary least squares regression over the selected time samples, where Cᵢ(Pₖ) represents a local regional compliance at pressure Pₖ.

5. The method of claim 1, wherein determining the regional compliance at more than two different pressures comprises determining a relationship between variation of the electrical impedance tomography data acquired in a time window only during a deflation part of the pressure-volume maneuver and a corresponding variation of pressure during the same period.

6. The method of claim 1, wherein calculating the cumulative overdistension and the cumulative collapse at each pressure of the more than two different pressures comprises:
determining the occurrence of overdistension if a selected pressure is higher than P_{maxC} or collapse if the selected pressure is lower than P_{maxC};
determining a weight for the lung region as a percent change in compliance of the lung region at the selected pressure relative to the compliance of the lung region at P_{maxC};
estimating the cumulative overdistension as a weighted average of all lung regions in which overdistension was determined at the selected pressure; and
estimating the cumulative collapse as a weighted average of all lung regions in which collapse was determined at the selected pressure.

7. The method of claim 1, wherein outputting a pressure P_{med} at which curves representing the cumulative overdistension and the cumulative collapse over the more than two different pressures intersect comprises at least one of:
outputting a marker corresponding to P_{med} on a graphical representation;
generating a line corresponding to P_{med} on a graphical representation;
outputting a numerical value corresponding to P_{med}; and
identifying and displaying a recommended pressure range based on the intersection to provide guidance for setting positive end-expiratory pressure levels.

8. The method of claim 7, further comprising causing a mechanical ventilator to set a positive end-expiratory pressure at or near the pressure P_{med}.

9. The method of claim 1, wherein acquiring the electrical impedance tomography data during the pressure-volume maneuver comprises deflating lungs of the mechanically ventilated patient at a rate within a range of from about 2 cmH₂O/s to about 5 cmH₂O/s, from a pressure greater than 30 cmH₂O to a pressure lower than 15 cmH₂O.

10. The method of claim 1, wherein at least one lung region of the plurality of lung regions is comprised entirely by non-dependent regions of the lung and at least another lung region of the plurality of lung regions is comprised entirely by dependent regions of the lung.

11. The method of claim 1, wherein the pressure values from the pressure-volume maneuver correspond to alveolar pressures calculated according to
$${P}_{alv} ( t ) \approx {P}_{aw} ( t ) - \dot{V} ( t ) \cdot {R}_{sys}$$
where P_{aw} is airway pressure, R_{sys} is respiratory system resistance, and \dot{V}(t) is airway flow.

12. The method of claim 1, wherein determining the regional compliance comprises fitting a sigmoidal model to the pressure-impedance curve.

13. The method of claim 1, wherein determining the regional compliance comprises determining the regional compliance in an absence of breathing cycles.

14. A system for assessing regional lung compliance in a mechanically ventilated patient, the system comprising:
an electrical impedance tomography acquisition system configured to acquire electrical impedance tomography data representative of a plurality of lung regions while a pressure-volume maneuver is performed on the mechanically ventilated patient;
one or more sensors or interfaces configured to obtain pressure values from the pressure-volume maneuver; and
a processor configured to:
associate the pressure values with corresponding regional electrical impedance tomography data to obtain a respective pressure-impedance curve;
determine regional compliance at more than two different pressures;
determine a pressure P_{maxC} at which the regional compliance is greatest;
determine occurrence of at least one of overdistension at pressures above P_{maxC} and collapse at pressures below P_{maxC};
calculate cumulative overdistension and cumulative collapse based on the occurrence of overdistension and collapse at the plurality of lung regions at each of the more than two different pressures; and
output a pressure P_{med} at which curves representing the cumulative overdistension and the cumulative collapse over the more than two different pressures intersect.

15. The system of claim 14, wherein the processor is further configured to determine the regional compliance at more than two different pressures by determining a slope of the regional pressure-impedance curve at each of the more than two different pressures.

16. The system of claim 14, wherein the processor is further configured to determine the regional compliance at the more than two different pressures by determining a relationship between variation of the electrical impedance tomography data acquired in a time window only during a deflation part of the pressure-volume maneuver and a corresponding variation of pressure during the same period.

17. The system of claim 14, wherein the processor is further configured to calculate the cumulative overdistension and the cumulative collapse at a certain pressure by:
determining the occurrence of overdistension if the selected pressure is higher than P_{maxC} or collapse if the selected pressure is lower than P_{maxC};
determining a weight for the lung region as a percent change in compliance of the lung region at the selected pressure relative to the compliance of the lung region at P_{maxC};
estimating the cumulative overdistension as a weighted average of all lung regions in which overdistension was determined at the selected pressure; and
estimating the cumulative collapse as a weighted average of all lung regions in which collapse was determined at the selected pressure.

18. The system of claim 14, wherein the processor is further configured to at least one of:
output a marker corresponding to P_{med} on a graphical representation;
generate a line corresponding to P_{med} on a graphical representation;
output a numerical value corresponding to P_{med}; and
identify and display a recommended pressure range based on the intersection to provide guidance for setting positive end-expiratory pressure levels.

19. The system of claim 14, wherein the processor is further configured to acquire electrical impedance tomography data representative of a plurality of lung regions, wherein at least one lung region of the plurality of lung regions is comprised entirely by non-dependent regions of a lung and at least another lung region of the plurality of lung regions is comprised entirely by dependent regions of the lung.

20. The system of claim 14, wherein the processor is further configured to adjust the pressure values from the pressure-volume maneuver to correspond to alveolar pressures calculated according to
$${P}_{alv} ( t ) \approx {P}_{aw} ( t ) - \dot{V} ( t ) \cdot {R}_{sys}$$
where P_{aw} is the airway pressure, R_{sys} is Respiratory System Resistance and \dot{V}(t) is airway flow.

21. The system of claim 14, wherein the processor is further configured to determine regional compliance by fitting a sigmoidal curve to the respective pressure-impedance curve.

22. The system of claim 14, wherein the processor is further configured to determine the regional compliance based on the respective pressure-impedance curve in an absence of breathing cycles.

23. The system of claim 14, further comprising a mechanical ventilator communicatively coupled to the processor, wherein the processor is configured to cause the mechanical ventilator to set a positive end-expiratory pressure at or near the pressure P_{med}.
